(19) United States
(12) Patent Application Publication
(10) Pub. No.: US 2023/0178250 A1
(43) Pub. Date: Jun. 8, 2023

(54) A METHOD FOR INFERRING EPILEPTOGENICITY OF A BRAIN REGION

(71) Applicants: UNIVERSITÉ D'AIX-MARSEILLE (AMU), Marseille (FR); INSTITUT NATIONAL DE LA SANTÉ ET DE LA RECHERCHE MÉDICALE, Paris (FR)

(72) Inventors: Viktor Jirsa, Aubagne (FR); Meysam Hashemi, Marseille (FR); Michael Marmaduke Woodman, Marseille (FR); Viktor Sip, Marseille (FR); Anirudh Nihalani Vattikonda, Marseille (FR)

(73) Assignees: UNIVERSITÉ D'AIX-MARSEILLE (AMU), Marseille (FR); INSTITUT NATIONAL DE LA SANTÉ ET DE LA RECHERCHE MÉDICALE, Paris (FR)

(21) Appl. No.: 17/916,128

(22) PCT Filed: Apr. 3, 2020
(86) PCT No.: PCT/EP2020/059511

§ 371 Date: Sep. 30, 2022

(51) Int. Cl. G16H 50/50; G06N 5/04

(52) U.S. Cl. CPC G16H 50/50; G06N 5/04

(57) ABSTRACT

The method for inferring epileptogenicity of a brain region not observed as recruited or not observed as not recruited, in a seizure activity of an epileptic patient brain, includes: providing a computerized model modelling various regions of a primate brain and connectivity between the regions; providing the computerized model with a model able to reproduce an epileptic seizure dynamic in the primate brain; providing structural data of the epileptic patient brain and personalizing the computerized model using the structural data to obtain a virtual epileptic patient (VEP) brain model; translating a state-space representation of the VEP brain model into a probabilistic programming language (PPL) using probabilistic state transitions to obtain a probabilistic virtual epileptic patient brain model (BVEP); and acquiring electro- or magneto-encephalographic data of the patient brain and fitting the probabilistic VEP brain model against the data to infer the epileptogenicity of the brain region that is not observed.

### FIELD OF THE INVENTION

The invention relates to a probabilistic method for inferring epileptogenicity of a brain region that is not observed as recruited or not observed as not recruited in a seizure activity of an epileptic patient brain.

### BACKGROUND OF THE INVENTION

Model inversion i.e., finding a set of model parameters that yields the best possible fit to the observed data is a challenging task in statistical inference. Bayesian frameworks offer powerful and principled methods for parameter inference and model prediction from experimental data with a broad range of applications. Within neuroimaging context, the Bayesian approaches have been widely used for inference of neuronal population’s intrinsic parameters and/or interactions between neuronal populations in a pre-specified neuronal network from neurophysiological data.

It is well-known that gradient-free sampling algorithms such as Metropolis-Hastings, Gibbs sampling and slice-sampling generally fail to explore the parameter space efficiently when applied to large-scale inverse problems, as often encountered in the application of whole-brain imaging for clinical diagnoses. In particular, the traditional Markov Chain Monte Carlo (MCMC) mix poorly in high-dimensional parameter spaces involving correlated variables. In contrast, gradient-based algorithms such as

Hamiltonian Monte Carlo (HMC), although computationally expensive, are far superior to gradient-free sampling algorithms in terms of the number of independent samples produced per unit computational time. This class of sampling algorithms provides efficient convergence and exploration of parameter space even in very high-dimensional spaces that may exhibit strong correlations. Nevertheless, the efficiency of gradient-based sampling methods such as HMC is highly sensitive to the user-specified algorithm parameters. More advanced MCMC sampling algorithms such as No-U-Turn Sampler (NUTS) a self-tuning variant of HMC solve these issues by adaptively tuning the algorithm parameters. It has been shown that these algorithms efficiently sample from high-dimensional target distributions that allow us to solve complex inverse problems conditioned on massive data set as the observation.

MCMC has the advantage of being non-parametric and asymptotically exact in the limit of long/infinite runs. Of the other alternatives, Variational Inference (VI) turns the Bayesian inference into an optimization problem, which typically results in much faster computation than MCMC methods. However, the classical derivation of VI requires a major model-specific work on defining a variational family appropriate to the probabilistic model, computing the corresponding objective function, computing gradients, and running a gradient-based optimization algorithm. Automatic Differentiation Variational Inference (ADVI) solves these problems automatically.

Probabilistic programming languages (PPLs) provide efficient implementation for automatic Bayesian inference on user-defined probabilistic models by featuring the next generation of MCMC sampling and VI algorithms such as NUTS and ADVI, respectively. With the help of PPLs, these algorithms take the advantage of automatic differentiation methods for the computation of derivatives in computer programs to avoid the random walk behaviour and sensitivity to correlated parameters. In particular, Stan and PyMC3 are high-level statistical modeling tools for Bayesian inference and probabilistic machine learning, which provide the advanced inference algorithms such as NUTS and ADVI, enriched with extensive and reliable diagnostics. Although PPLs allow for automatic inference, the performance of these algorithms can be sensitive to the form of parameterization. An appropriate form of reparameterization in the probabilistic models to improve the inference efficiency of system dynamics (governed by a set of nonlinear stochastic differential equations) remains a challenging problem.

On the other hand, due to the potential to improve medical treatment strategies, the personalized large-scale brain network modeling has gained popularity over the recent years. In the individualized whole-brain modeling approach, the patient-specific information such as anatomical connectivity obtained from non-invasive imaging techniques is combined with the mean-field models of local neuronal activity to simulate the individual’s spatiotemporal brain activity at the macroscopic scale. The Virtual Brain (TVB) is an open-access computational framework written in Python to reproduce and evaluate the personalized configurations of the brain by using individual subject data. This neuroinformatics platform integrates brain computational modeling and multimodal neuroimaging data to systematically simulate the individual’s spatiotemporal brain activity. However, there is currently no specific workflow for automatic model inversion and data fitting validation in preparation for TVB.

More recently, it was proposed a novel approach namely Virtual Epileptic Patient (VEP) to brain interventions based on personalized brain network models derived from non-invasive structural data of individual patients. The VEP model is a large-scale computational model of an individual brain that incorporates personal data such as the locations of seizure initiation, subject-specific brain connectivity, and MRI lesions to inform patient-specific clinical monitoring and improve surgical outcomes. It has been previously shown that the VEP model is able to realistically mimic the evolution of epileptic seizures in a patient with bitemporal epilepsy. However, the inverse problem of such large-scale brain network models is a challenging task due to the intrinsic non-linear dynamics of each brain network node as well as the related large number of model parameters and the observation as commonly encountered in brain-imaging setting.

### SUMMARY OF THE INVENTION

Accordingly, a need exists for establishing a useful link between the most popular probabilistic programming tools (e.g., Stan/PyMC3) and the personalized brain network modeling (e.g., the VEP model), in order to systematically predict the location of seizure initiation in a virtual epileptic patient. The present invention allows to build, in particular, a Bayesian Virtual Epileptic Patient (BVEP) as a probabilistic framework designed to infer the hidden/unobserved dynamics of personalized large-scale brain model of epilepsy spread generated by TVB.

In accordance with a first aspect, the invention concerns a method for inferring the epileptogenicity of a brain region that is not observed as recruited or is not observed as not recruited, in a seizure activity of an epileptic patient brain, comprising the steps of:
providing a computerized model modelling various regions of a primate brain and connectivity between said regions;
providing said computerized model with a model able to reproduce an epileptic seizure dynamic in the primate brain, said model being a function of a parameter that is the epileptogenicity of a region of the brain;
providing structural data of the epileptic patient brain and personalizing the computerized model using said structural data in order to obtain a virtual epileptic patient (VEP) brain model;
translating a state-space representation of the virtual epileptic patient (VEP) brain model into a probabilistic programming language (PPL) using probabilistic state transitions in order to obtain a probabilistic virtual epileptic patient brain model (BVEP); and
acquiring electro- or magneto- encephalographic data of the patient brain and fitting the probabilistic virtual epileptic patient brain model against said data in order to infer the epileptogenicity of said brain region that is not observed as recruited or not observed as not recruited, in the seizure activity of the patient brain.

Preferentially, – the probabilistic programming language is a Bayesian programming language, the probabilistic virtual epileptic patient brain model is Bayesian virtual epileptic patient (BVEP) brain model, and the epileptogenicity of the brain region that is not observed as recruited or not observed as not recruited, is inferred using Bayesian inference; – the structural data of the epileptic patient brain comprise non-invasive T1-weighted imaging data and/or diffusion MRI images data; – the model able to reproduce the epileptic seizure dynamic in the primate brain is a model which reproduces the dynamics of an onset, a progression and an offset seizure events, that comprises state variables coupling two oscillatory dynamical systems on three different timescales, a fastest timescale wherein state variables account for fast discharges during an ictal seizure state, an intermediate timescale wherein state variables represent slow spike-and-wave oscillation, and a slowest timescale wherein a state variable is responsible for the transition between interictal and ictal states, and wherein a degree of epileptogenicity of a region of the brain is represented through a value of an excitability parameter; – for obtaining the probabilistic virtual epileptic patient brain model, a spatial map of epileptogenicity of the patient brain is provided, said spatial map of epileptogenicity classifying brain regions of the patient brain into epileptogenic zones (EZ) which can trigger epileptic seizures autonomously, propagation zones (PZ) which do not trigger seizures autonomously but can be recruited during a seizure evolution, and healthy zones (HZ) that do not trigger seizures autonomously; – the probabilistic virtual epileptic patient brain model is generated according to a generative model based on the state-space representation of the virtual epileptic patient; – the state-space representation of the virtual epileptic patient is of the form

$$\left\{\begin{matrix}\dot{x} \left(t\right) = f \left(x \left(t\right) , u \left(t\right) , θ\right) + w \left(t\right) , x \left(0\right) = {x}_{{t}_{0}} \\ y \left(t\right) = h \left(x \left(t\right)\right) + v \left(t\right)\end{matrix}\right)$$

where x(t) ∈ ℝⁿ is a n-dimensional vector of system’s states evolving over time, xₜ₀ is an initial state vector at time t = 0, θ ∈ ℝ^{p} contains all the unknown parameters of the virtual epileptic patient model, u(t) stands for the external input, y(t) ∈ ℝ^{m} denotes the measured data subject to the measurement error v(t), f is a vector function that describes dynamical properties of the system and h represents a measurement function; – for obtaining of the probabilistic virtual epileptic patient (BVEP) model, the state-space representation of the virtual epileptic patient (VEP) model is incorporated in the probabilistic virtual epileptic patient (BVEP) model as state transition probabilities; – the state transition probabilities are such as:

$$T \left(x \left(t\right) , x \left(t + 1\right)\right) \sim N \left(x \left(t\right) + d t f \left(x \left(t\right) , u \left(t\right) , θ\right) , {σ}^{2}\right)$$

where T denotes the transition probability from a state x(t) to x(t + dt); – the generative model is defined in terms of likelihood and prior on model parameters, whose product yields a joint density:

$$P \left(Y , ϑ\right) = P \left(Y \vert ϑ\right) P \left(ϑ\right)$$

where prior distribution P(ϑ) includes our prior beliefs about the hidden variables and potential parameter values, while the conditional likelihood term P(γ|ϑ) represents the probability of obtaining an observation, with a given set of parameter values; – in order to infer the epileptogenicity of the brain region that is not observed as recruited or not observed as not recruited, in the seizure activity of the patient brain, sampling algorithms are implemented; – the sampling algorithm is a Markov chain Monte Carlo or a variational inference algorithm; and – the method is computer implemented.

### BRIEF DESCRIPTION OF THE DRAWINGS

Other features and aspects of the present invention will be apparent from the following description and the accompanying drawings, in which:

FIG. 1 is a schematic illustration of the method according to the invention;

FIGS. 2A, 2B, 2C, 2D and 2E illustrate the results that are obtained according to the method of the invention, for estimating the spatial map of epileptogenicity across different brain regions for a patient. More particularly, FIG. 2A illustrates the parcellation of reconstructed brain of the patient, FIG. 2B illustrates a brain network of a patient consisting of 84 regions (grey: HZ, light grey: PZ, dark grey: EZ). Thickness of the lines indicates the strength of the connections. For illustration purposes, only connections with weight above 10% of the maximum weight are shown. FIG. 2C illustrates a structural connectivity matrix. FIG. 2D Exemplary simulation of full VEP model at the source-level brain activity versus the predicted envelope (dashed line). FIG. 2E illustrates the estimated densities of the excitability parameters ηᵢ for different brain node types, and wherein the vertical dashed lines indicate the true values;

FIGS. 3A, 3B, 3C, 3D and 3E illustrate the accuracy of the results that are obtained according to the method of the invention, as concerns the estimated spatial map of epileptogenicity across different brain regions using the NUTS algorithm for a patient 1. More particularly, FIG. 3A illustrates an example of observed data (dash-dotted lines) versus the prediction for three brain node types defined as HZ (grey), PZ (light grey), and EZ (dark grey). The shaded area depicts the ranges between the 5th and 95th percentiles of the posterior predictive distribution. FIG. 3B shows plots indicative of the estimated densities of the ηᵢ for 84 brain regions. The true values are displayed by the filled black circles. FIG. 3C illustrates the distribution of posterior z-scores versus posterior shrinkages, that implies an ideal Bayesian inversion. FIG. 3D shows a confusion matrix of the estimated spatial map of epileptogenicity. The pre-defined class for all the brain nodes labelled as HZ, PZ, and EZ are accurately predicted (accuracy=1.0, misclass=0.0);

FIG. 4 illustrates a comparison between the simulated (top row) and the predicted (bottom row) phase-plane for different brain node types in the BVEP model. From left to right, the columns correspond to the brain nodes specified as HZ, PZ, and EZ, respectively. A trajectory for these brain regions is shown in green, yellow and red, respectively. In each phase-plane, depending on the excitability parameter, the intersection of x- and z-nullclines (coloured in dark grey) determines the fixed point of the system. Full circle and empty circle indicate the stable and unstable fixed points, respectively;

FIG. 5 illustrates an estimated spatial map of epileptogenicity obtained by NUTS algorithm in comparison to ADVI. Exemplary histogram and the kernel density estimates of the samples obtained by NUTS are illustrated in panel A versus the approximation by mean-field variant of ADVI shown in panel B. For all the brain nodes included in the analysis, the prior (shown in dark grey) was assumed as N(-2.5, 1.0). The dashed vertical lines indicate the true values; and

FIG. 6 illustrates the NUTS and ADVI convergence diagnostics. In panel A, it is shown the samples generated by NUTS from joint posterior probability distribution between the hyperparameter pair (σ, σ′). In this case, the non-centered form of parameterization yields independent samples from the posterior distribution. In panel B The centered form of sampling leads to high correlation between hyper-parameters indicating that the sampler was not efficiently exploring the posterior distribution. In panel C the samples from approximate joint posterior probability distribution using the mean-field variant of ADVI. Panel D shows the R values for the non-centered form of sampling, which are lower than 1.05 for all estimated hidden states and parameters implying that the MCMC has converged. In panel E, the high values of R̂ returned by the centered form of sampling indicate that the chain has not converged. In panel F the variational objective function (ELBO) versus the number of ADVI iterations.

### DETAILLED DESCRIPTION OF THE INVENTION

The invention relates to a method for inferring an epileptogenicity of a brain region that is not observed as recruited or not observed as not recruited in a seizure activity of an epileptic patient brain. This is a computerized probabilistic method for inferring the spatial map of epileptogenicity across different brain regions in a personalized epileptic brain patient that the seizures initiate in hypothetical areas and may propagate to candidate brain regions.

The method according to the invention comprises various steps that are computer implemented. A computer readable medium is encoded with computer readable instructions for performing the steps of the method according to the invention.

It comprises a step of providing a computerized model modelling various regions of a primate brain and connectivity between said regions. This brain is a virtual brain. It is a neuro-informatics platform for full brain network simulations using biologically realistic connectivity. This simulation environment enables the model-based inference of neurophysiological mechanisms across different brain scales that underlie the generation of macroscopic neuroimaging signals including functional Magnetic Resonance Imaging (fMRI), EEG and Magnetoencephalography (MEG). It allows the reproduction and evaluation of personalized configurations of the brain by using individual subject data.

It further comprises a step of providing said computerized model with a model able to reproduce an epileptic seizure dynamic in the primate brain, said model being a function of a parameter that is the epileptogenicity of a region of the brain.

Preferentially, the model able to reproduce the epileptic seizure dynamic in the primate brain is a model which reproduces the dynamics of an onset, a progression and an offset seizure events, that comprises state variables coupling two oscillatory dynamical systems on three different timescales, a fastest timescale wherein state variables account for fast discharges during an ictal seizure state, an intermediate timescale wherein state variables represent slow spike-and-wave oscillation, and a slowest timescale wherein a state variable is responsible for the transition between interictal and ictal states, and wherein a degree of epileptogenicity of a region of the brain is represented through a value of an excitability parameter.

Also, according to the invention comprises a step of providing structural data of the epileptic patient brain and personalizing the computerized model using said structural data in order to obtain a virtual epileptic patient (VEP) brain model. The structural data are, for example, images data of the patient brain acquired using magnetic resonance imaging (MRI), diffusion-weighted magnetic resonance imaging (DW-MRI), nuclear magnetic resonance imaging (NMRI), or magnetic resonance tomography (MRT). Preferentially, the structural data of the epileptic patient brain comprise non-invasive T1-weighted imaging data and/or diffusion MRI images data.

The method according to the invention further comprises a step of translating a state-space representation of the virtual epileptic patient (VEP) brain model into a probabilistic programming language (PPL) using probabilistic state transitions in order to obtain a probabilistic virtual epileptic patient brain model (BVEP).

Preferentially, the probabilistic programming language is a Bayesian programming language, the probabilistic virtual epileptic patient brain model is Bayesian virtual epileptic patient (BVEP) brain model, and the epileptogenicity of the brain region that is not observed, neither as recruited nor as not recruited, is inferred using Bayesian inference.

Preferentially, for obtaining the probabilistic virtual epileptic patient brain model, a spatial map of epileptogenicity of the patient brain is provided, said spatial map of epileptogenicity classifying brain regions of the patient brain into epileptogenic zones (EZ) which can trigger epileptic seizures autonomously, propagation zones (PZ) which do not trigger seizures autonomously but can be recruited during a seizure evolution, and healthy zones (HZ) that do not trigger seizures autonomously.

Preferentially, the probabilistic virtual epileptic patient brain model is generated according to a generative model based on the state-space representation of the virtual epileptic patient.

More preferentially, the state-space representation of the virtual epileptic patient is of the form

$$\left\{\begin{matrix}\dot{x} \left(t\right) = f \left(x \left(t\right) , u \left(t\right) , θ\right) + w \left(t\right) , x \left(0\right) = {x}_{{t}_{0}} \\ y \left(t\right) = h \left(x \left(t\right)\right) + v \left(t\right)\end{matrix}\right)$$

where x(t) ∈ ℝⁿ is a n-dimensional vector of system’s states evolving over time, xₜ₀ is an initial state vector at time t = 0, θ ∈ ℝ^{p} contains all the unknown parameters of the virtual epileptic patient model, u(t) stands for the external input, y(t) ∈ ℝ^{m} denotes the measured data subject to the measurement error v(t), f is a vector function that describes dynamical properties of the system and h represents a measurement function.

Preferentially, for obtaining of the probabilistic virtual epileptic patient (BVEP) model the state-space representation of the virtual epileptic patient (VEP) model is incorporated in the probabilistic virtual epileptic patient (BVEP) model as state transition probabilities.

More preferentially, the state transition probabilities are such as:

$$T \left(x \left(t\right) , x \left(t + 1\right)\right) \sim N \left(x \left(t\right) + d t f \left(x \left(t\right) , u \left(t\right) , θ\right) , {σ}^{2}\right)$$

where T denotes the transition probability from a state x(t) to x(t + dt) .

More preferentially, the generative model is defined in terms of likelihood and prior on model parameters, whose product yields a joint density:

$$P \left(Y , ϑ\right) = P \left(Y \vert ϑ\right) P \left(ϑ\right)$$

where prior distribution P(ϑ) includes our prior beliefs about the hidden variables and potential parameter values, while the conditional likelihood term P(γ|ϑ) represents the probability of obtaining an observation, with a given set of parameter values.

The method according to the invention further comprises the step of acquiring electro- or magneto-encephalographic data of the patient brain and fitting the probabilistic virtual epileptic patient brain model against said data in order to infer the excitability of said brain region that is not observed, neither as recruited nor as not recruited, in the seizure activity of the patient brain.

Preferentially, in order to infer the excitability of the brain region that is not observed as recruited or not observed as not recruited, in the seizure activity of the patient brain, sampling algorithms are implemented.

More preferentially, the sampling algorithm is a Markov chain Monte Carlo or a variational inference algorithm.

### Example 1: Materials and Methods

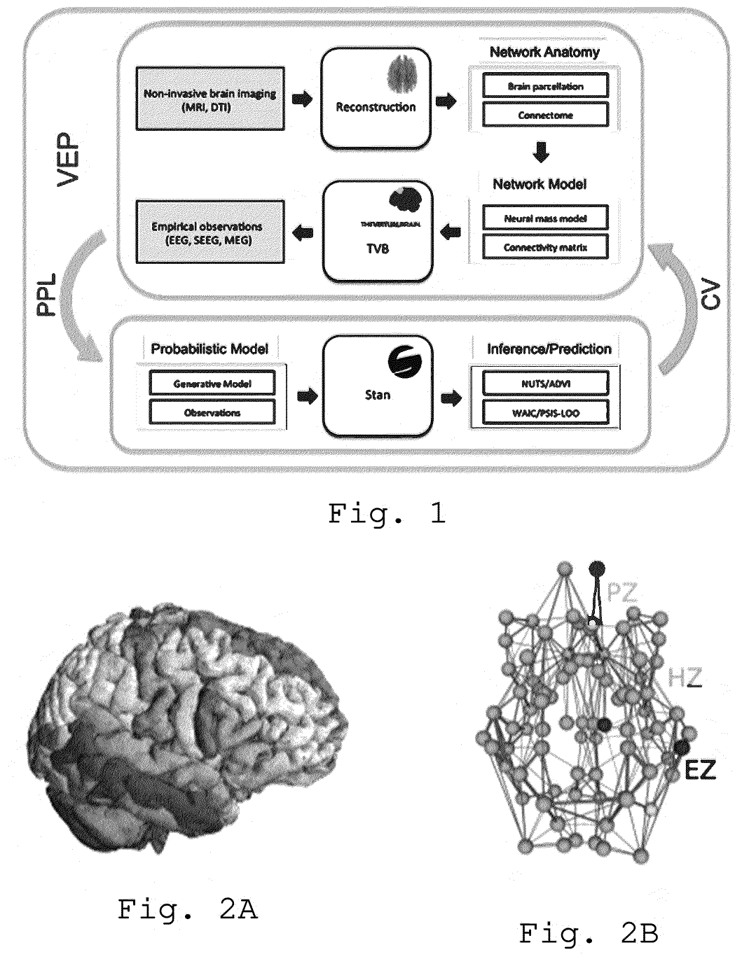

In an example, the method according to the invention is based on the personalized brain network modeling and Bayesian inference as schematically illustrated in FIG. 1. This method allows to build the BVEP according to two main steps, which are constructing the VEP model, and then embedding VEP in a PPL tool to infer and validate the model parameters. To build the VEP model, the following steps are carried out: first, the patient undergoes non-invasive brain imaging (MRI, DTI). Based on these images, the brain network anatomy, including brain parcellation and the patient’s connectome, are provided from the reconstruction pipeline. Then, a neural population model is selected for each brain region to define the network model. In the VEP, the Epileptor™ model is defined on each network node that are connected through structural connectivity derived from diffusion tractography. Such a model is disclosed, for example, in the publication document entitled “On the nature of seizure dynamics”, Jirsa et al., Brain 2014, 137, 2210-2230, which is incorporated herein, by citation of reference. It comprises five state variables acting on three different time scales. On the fastest time scale, state variables account for the fast discharges during the seizure. On the slowest time scale, the permittivity state variable accounts for slow processes such as variation in extracellular ion concentrations, energy consumption, and tissue oxygenation. The system exhibits fast oscillations during the ictal state through the state variables. Autonomous switching between interictal and ictal states is realized via the permittivity state variable through saddle-node and homoclinic bifurcation mechanisms for the seizure onset and offset, respectively. The switching is accompanied by a direct current (DC) shift, which has been recorded in vitro and in vivo. On the intermediate time scale, other state variables describe the spike-and-wave electrographic patterns observed during the seizure, as well as the interictal and preictal spikes when excited by the fastest system via coupling. Put together, TVB simulations allow to mimic the empirical neuroimaging signals. Then, model fitting is performed using, for example, NUTS/ADVI algorithms within a PPL tool (in this example, the brain source activity as the observation, and the VEP model as the generative model translated in Stan/PyMC3). Finally, cross validation can be performed, for example, by WAIC/LOO from the existing samples to assess the model’s ability in new data prediction, thus, in order to refine the network pathology. In other words, the workflow to build the BVEP consists of two main steps: constructing the VEP, a personalized brain network model of epilepsy spread, and then embedding the VEP model in a Bayesian framework to infer and validate the model parameters. Following the VEP formulation in state-space representation, the probabilistic reparameterization of the system dynamics is demonstrated. It is shown that the proposed probabilistic reparameterization in BVEP is able to efficiently invert the nonlinear state-space equations to infer the system dynamics. This approach allows to accurately estimate the spatial map of epileptogenicity in a personalized brain network model of epilepsy spread by taking advantage of PPLs. The virtual brain is used for brain network simulations, and Stan as well as PyMC3 for inverting the simulated whole-brain model.

In what follows, it is shown step by step how to build the BVEP model for a particular patient in order to fit the constructed brain model against in-silico data and validate our inference. The accuracy and the reliability of the estimations are validated by several convergence diagnostics and posterior behaviour analysis.

### Individual Patient Data

For this study, two patients were selected: a 23 year-old female with drug-resistant occipital lobe epilepsy (patient 1), and a 24 year-old female with drug-resistant temporo-frontal lobe epilepsy (patient 2). The patients underwent standard clinical evaluation, details of which were described in a previous study (Proix et al., Individual brain structure and modelling predict seizure propagation. Brain 140, 641-654, 2017). The evaluation included non-invasive T1-weighted imaging (MPRAGE sequence, repetition time = 1900 ms, echo time = 2.19 ms, 1.0 × 1.0 × 1.0 mm, 208 slices) and diffusion MRI images (DTI-MR sequence, angular gradient set of 64 directions, repetition time = 10.7 s, echo time = 95 ms, 2.0 × 2.0 × 2.0 mm, 70 slices, b-weighting of 1000 s mm⁻²). The images were acquired on a Siemens Magnetom Verio™ 3T MR-scanner.

### Network Anatomy

The structural connectome was built with a reconstruction pipeline using generally available neuroimaging software. The current version of the pipeline evolved from a previously described version (Proix et al., 2017). First, the command recon-all from Freesurfer™ package in version v6.0.0 was used to reconstruct and parcellate the brain anatomy from T1-weighted images. Then, the T1-weighted images were coregistered with the diffusion weighted images by the linear registration tool flirt™ from FSL package in version 6.0 using the correlation ratio cost function with 12 degrees of freedom.

The MRtrix™ package in version 0.3.15 was then used for the tractography. The fibre orientation distributions were estimated from DWI using spherical deconvolution by the dwi2fod tool with the response function estimated by the dwi2response tool using the Tournier algorithm. Next, the tckgen tool was used, employing the probabilistic tractography algorithm iFOD2 to generate 15 millions fiber tracts. Finally, the connectome matrix was built by the tck2connectome tool using the Desikan-Killiany parcellation generated by FreeSurfer in the previous step. The connectome was normalized so that the maximum value is equal to one.

### Network Model

Typically, to build a personalized brain network model, the brain regions are defined using a parcellation scheme and a set of mathematical equations is used to model the regional brain activity. Taking such a data-driven approach to incorporate the subject-specific brain’s anatomical information, the network edges are then represented by structural connectivity of the brain, which are obtained from non-invasive imaging data of individual patients. In the VEP model, the dynamics of brain network nodes are governed by the Epileptor equations (Jirsa et al., On the nature of seizure dynamics. Brain 137, 2210-2230, 2014) that are coupled through the structural connectivity matrix derived from diffusion-weighted MRI (dMRI) techniques (Jirsa et al., The virtual epileptic patient: Individualized whole-brain models of epilepsy spread, 2017) .

The Epileptor is a dynamical model of seizure evolution and is able to realistically reproduce the dynamics of onset, progression and onset of seizure-like events. The Epileptor comprises five state variables coupling two oscillatory dynamical systems on three different timescales: on the fastest timescale, variables x₁ and y₁ account for fast discharges during the ictal seizure states. On the intermediate timescale, variables x₂ and y₂ represent the slow spike-and-wave oscillations. On the slowest timescale, the permittivity state variable z is responsible for the transition between interictal and ictal states. In addition, the interictal and preictal spikes are generated via the term g(x₁).

Following Jirsa et al. (2014), the dynamics of full Epileptor model is described by:

$$\begin{matrix}{\dot{x}}_{1} = {y}_{1} - {f}_{1} \left({x}_{1} , {x}_{2}\right) - z + {I}_{1} \\ {\dot{y}}_{1} = \frac{1}{{τ}_{1}} \left(1 - 5 {x}_{1}^{2} - {y}_{1}\right) \\ \dot{z} = \frac{1}{{τ}_{0}} \left(4 \left({x}_{1} - η\right) - z\right) \\ {\dot{x}}_{2} = - {y}_{2} + {x}_{2} - {x}_{2}^{3} + {I}_{2} + 0.002 g \left({x}_{1}\right) - 0.3 \left(z - 3.5\right) \\ {\dot{y}}_{2} = \frac{1}{{τ}_{2}} \left(- {y}_{2} + {f}_{2} \left({x}_{2}\right)\right)\end{matrix}$$

where

$${f}_{1} \left({x}_{1} , {x}_{2}\right) = \left\{\begin{matrix}{x}_{1}^{3} - 3 {x}_{1}^{2} & \text{if} {x}_{1} < 0 \\ \left({x}_{2} - 0.6 {\left(z - 4\right)}^{2}\right) {x}_{1} & \text{if} {x}_{1} \geq 0\end{matrix}\right)$$

$${f}_{2} \left({x}_{2}\right) = \left\{\begin{matrix}0 & \text{if} {x}_{2} < - 0.25 \\ 6 \left({x}_{2} + 0.25\right) & \text{if} {x}_{2} \geq - 0.25\end{matrix}\right)$$

$$g \left({x}_{1}\right) = {\int }_{- {t}_{0}}^{t} {exp}^{- γ \left(t - τ\right)} {x}_{1} \left(τ\right) d t ,$$

with τ₀ = 2857, τ₁ = 1, τ₂ = 10, I₁ = 3.1, I₂ = 0.45, and γ = 0:01. The degree of epileptogenicity is represented through the value of excitability parameter η. If η > ^{η}c, where ^{η}c is the critical value of epileptogenicity, Epileptor shows seizure activity autonomously and is referred to as epileptogenic; otherwise Epileptor is in its (healthy) equilibrium state and does not trigger seizures autonomously.

Following Jirsa et al. (2017), the full VEP brain model equations (N-coupled Epileptors) read as follows:

$$\begin{matrix}{\dot{x}}_{1 , i} = {y}_{1 , i} - {f}_{1} \left({x}_{1 , i} , {x}_{2 , i}\right) - {z}_{i} + {I}_{1} \\ {\dot{y}}_{1 , i} = \frac{1}{{τ}_{1}} \left(1 - 5 {x}_{1 , i}^{2} - {y}_{1 , i}\right) \\ {\dot{z}}_{i} = \frac{1}{{τ}_{0}} \left(4 \left({x}_{1 , i} - {η}_{i}\right) - {z}_{i} - K {\sum }_{j = 1}^{N} {C}_{i j} \left({x}_{1 , j} - {x}_{1 , i}\right)\right) \\ {\dot{x}}_{2 , i} = - {y}_{2 , i} + {x}_{2 , i} - {x}_{2 , i}^{3} + {I}_{2} + 0.002 g \left({x}_{1 , i}\right) - 0.3 \left({z}_{i} - 3.5\right) \\ {\dot{y}}_{2 , i} = \frac{1}{{τ}_{2}} \left(- {y}_{2 , i} + {f}_{2} \left({x}_{2 , i}\right)\right)\end{matrix}$$

where the network nodes are coupled by a linear approximation of permittivity coupling through

$$K {\sum }_{j = 1}^{N} {C}_{i j} \left({x}_{1 , j} - {x}_{1 , i}\right) ,$$

which includes a global scaling factor K, and the patient’s connectome Cᵢⱼ.

Under the assumption of timescale separation, Proix et al. (2014) have shown that the effect of second neuronal ensemble of Epileptor (i.e., the variables x₂ and y₂) is negligible by averaging on the coupled Epileptor equations, which yields the 2D reduction of VEP model as follows:

$$\begin{matrix}{\dot{x}}_{1 , i} = 1 - {x}_{1 , i}^{3} - 2 {x}_{1 , i}^{2} - {z}_{i} + {I}_{1 , i} \\ {\dot{z}}_{i} = \frac{1}{τ} \left(4 \left({x}_{1 , i} - {η}_{i}\right) - {z}_{i} - K {\sum }_{j = 1}^{N} {C}_{i j} \left({x}_{1 , j} - {x}_{1 , i}\right)\right) .\end{matrix}$$

Depending on the value of excitability parameter η, the 2D Epileptor exhibits different stability regimes. For η < ^{η}c, a trajectory in the phase plane is attracted to the single stable fixed point of the system on the left branch of the cubic x-nullcline. In this regime, the Epileptor is said to be healthy, meaning not triggering epileptic seizure without external input. As the value of η increases, the z-nullcline moves down and a saddle-node bifurcation occurs at η = ^{η}c corresponding to a seizure onset. For η > ^{η}c, the system exhibits an unstable fixed point allowing a seizure to happen (the Epileptor is said to be epileptogenic). In this example, we use the 2D reduction of VEP model is used for Bayesian inference of spatial map of epileptogenicity to reduce the computational cost associated with the model parameter estimation. The 2D reduction of Epileptor allows for faster inversion while enabling us to predict the envelope of fast discharges during the ictal seizure states (i.e., onset, propagation and offset of seizure patterns) (Proix et al., 2014; Jirsa et al., 2017).

### Spatial Map of Epileptogenicity

In addition to the patient’s connectome which structurally constraints the individual variability, the dynamics of brain network model can be further constrained by hypothesis formulation about functional brain network component to produce more specific patterns of brain activity across individuals. In the case of epilepsy, clinical hypothesis on the location of epileptogenic zone or lesion allows refining the network pathology to better predict seizure initialization and propagation in individual patients.

In the BVEP brain model, each network node can trigger seizures depending on its connectivity and the excitability value. The parameter η controls the tissue excitability, and its spatial distribution is thus the target of parameter fitting. In this study, depending on the excitability value, the different brain regions are classified into three main types:
Epileptogenic Zone (EZ): if η > ^{η}c, the Epileptor can trigger seizures autonomously (brain region responsible for the origin and early organization of the epileptic activity).
Propagation Zone (PZ) : if ^{η}c - Δ^{η} < ^{η} < ^{η}c, the Epileptor does not trigger seizures autonomously but they may be recruited during the seizure evolution since their equilibrium state is close to the critical value.
Healthy Zone (HZ): if ^{η} < ^{η}c - Δ^{η}, the Epileptor does not trigger seizures autonomously.

Based on the above dynamical properties, the spatial map of epileptogenicity across different brain regions comprises the excitability values of EZ (high value of excitability), PZ (smaller excitability values) and all other regions categorized as HZ (not epileptogenic). Note however, that an intermediate excitability value does not guarantee that the seizure recruits this area as part of the propagation zone, because the propagation is also determined by various other factors including connectivity and brain state dependence. In the BVEP brain model, the clinical hypotheses can be formulated as the prior knowledge on the spatial distribution of excitability parameters. In this study, assuming no clinical hypothesis on a particular brain area, the same prior distribution were assigned on the excitability parameter across all brain regions included in the analysis.

### Probabilistic Model

The key component in constructing a probabilistic brain network model within a Bayesian framework is the generative model. Given a set of observations, the generative model is a probabilistic description of the mechanisms by which observed data are generated through some hidden states and unknown parameters. Here, the generative model will therefore have a mathematical formulation guided by the dynamical model that describes the evolution of model’s state variables, given parameters, over time. This specification is necessary to construct the likelihood function. The full generative model is then completed by specifying prior beliefs about the possible values of the unknown parameters.

The BVEP brain model presented in this study is built upon two main steps. First, the VEP model equation that provides the basic form of the data generative process describing how the epileptic seizures are generated. Second, the hypothesis formulation on the spatial map of epileptogenicity in the brain as our prior knowledge. The later component informs the model using hypotheses about the spatial distribution of excitability parameter across different brain regions.

The generative model in the BVEP is formulated based on a system of nonlinear stochastic differential equations of the form (so-called state-space representation):

$$\left\{\begin{matrix}\dot{x} \left(t\right) = f \left(x \left(t\right) , u \left(t\right) , θ\right) + w \left(t\right) , x \left(0\right) = {x}_{{t}_{0}} \\ y \left(t\right) = h \left(x \left(t\right)\right) + v \left(t\right)\end{matrix}\right)$$

where x(t) ∈ ℝⁿ is a n-dimensional vector of system’s states evolving over time, xₜ₀ is an initial state vector at time t = 0, θ ∈ ℝ^{p} contains all the unknown parameters of the virtual epileptic patient model, u(t) stands for the external input, y(t) ∈ ℝ^{m} denotes the measured data subject to the measurement error v(t). The process (dynamical) noise and the measurement noise denoted by w(t) ~ N(0; σ²) and v(t) ~ N(0; σ′²), respectively, are assumed to follow a Gaussian distribution with mean zero and variance σ² and σ′², respectively. The coloured and non-Gaussian dynamical noise can be captured in the term w(t), whereas in the presence of multiplicative noise (i.e., the noise whose intensity depends upon the system’s state) or multiplicative feedback (the system’s state further influences the driving noise intensity), an additional term appears which can lead to qualitatively different solutions. Moreover, f(.) is a vector function that describes the dynamical properties of the system and h(.) represents a measurement function. In source localization problem, h(.) is known as the lead-field matrix. It is noted that the current work focuses on the potential brain sources of observed activity to avoid the inevitable inconsistency associated with mapping from source dipoles to the measurements at electrode contacts (i.e., h(.) is a linear function here).

Considering the 2D reduction of VEP model (cf. Eq. (3)), then x(t) = (x_{1,1}, z₁, x_{1,2}, z₂, ..., x_{1,N}, z_{N}) ∈ ℝⁿ, with n = 2N, where N is equal to the number of brain regions. Accordingly, θ = (x_{t0,1}, x_{t0,2}, ...., x_{t0,N}, η₁, η₂, ..., η_{N}, K, σ, σ′) € ℝ^{p}, where p = 3N + 3. Using the reconstruction pipeline to virtualize a patient as described in section 2.2, here N = 84.

The state-space representation (cf. Eq. (4)) defining the dynamics of hidden states x(t) is incorporated in the BVEP model as state transition probabilities:

$$T \left(x \left(t\right) , x \left(t + 1\right)\right) \sim N \left(x \left(t\right) + d t f \left(x \left(t\right) , u \left(t\right) , θ\right) , {σ}^{2}\right)$$

where T denotes the transition probability from a state x(t) to x(t + dt). However, the above parameterization referred to as centered parameterization may exhibit a pathological geometry yielding biased estimations.

It has been previously shown that a careful choice of reparameterization increases the effective sample size and decreases the divergences, in particular for the regions of extreme curvature. To avoid pathological samples, and therefore, the biased estimations due to strong correlation between parameters in the centered form of parameterization, the advantage of location-scale transformation is taken to invert the nonlinear state-space equations, which allows to decorrelate the parameters representing state variables at successive time steps.

A non-centered reparameterization of the above distribution reads as follows:

$$\begin{matrix}T \left(x * \left(t\right) , x * \left(t + d t\right)\right) \sim N \left(0 , 1\right) , \\ x \left(t + d t\right) = f \left(x \left(t\right) , u \left(t\right) , θ\right) + d t d x + σ x * \left(t + d t\right) .\end{matrix}$$

In Example 3, it is shown that using the non-centered form of parameterization to infer the system dynamics dramatically improves the performance of sampling by avoiding biased estimations due to the strong correlation between parameter.

### Inference/Prediction

A generative model is characterized by the joint probability distribution of the model parameters and the observation P(y|ϑ) where Y denotes the observed variables, and ϑ includes the system’s hidden variables and the model parameters. Bayesian techniques infer the distribution of unknown parameters of the underlying data generating process, given only observed responses and prior beliefs about the underlying generative process. By product rule, the generative model can be defined in terms of likelihood and prior on the model parameters, whose product yields the joint density:

$$P \left(Y , ϑ\right) = P \left(Y \vert ϑ\right) P \left(ϑ\right) ,$$

where prior distribution P(ϑ) includes our prior beliefs about the hidden variables and potential parameter values, while the conditional likelihood term P(y|ϑ) represents the probability of obtaining the observation, with a given set of parameter values. In Bayesian inference, we seek the posterior density P(ϑ|y), which is the conditional distribution of model parameters given the observation. Bayes’s Theorem expresses this posterior density in terms of likelihood and prior as follows:

$$P \left(ϑ \vert Y\right) = \frac{P \left(Y \vert ϑ\right) P \left(ϑ\right)}{P \left(Y\right)} ,$$

where the denominator P(y) represents the probability of the data and it is known as evidence or marginal likelihood (in practice amounts to simply a normalization term).

To sample from posterior density P(ϑ|y), the performance of HMC is highly sensitive to the step size and the number of steps in leapfrog integrator for updating the position and momentum variables in Hamiltonian dynamic simulation. If the number of steps in the leapfrog integrator is chosen too small, then HMC exhibits an undesirable random walk behaviour similar to Metropolis-Hastings algorithm, and thus algorithm poorly explores the parameter space. If the number of leapfrog steps is chosen too large, the associated Hamiltonian trajectories may loop back to a neighbourhood of the initial state, and the algorithm wastes computation efforts. NUTS extends HMC with adaptive tuning of both the step size and the number of steps in leapfrog integration to sample efficiently from posterior distributions. In an alternative approach, ADVI posits a family of densities, automatically computes the gradients, and then finds the closest member (measured by Kullback-Leibler divergence) to the target distribution. In this study, we use NUTS, a self-tuning variant of HMC, as well as ADVI to approximate the posterior distribution of the model parameters (cf. Eq. (3)).

The prior on excitability parameter for all brain regions included in the analysis was assumed as a normal distribution with a mean of –2.5 and a standard deviation of 1.0, i.e., N(–2.5, 1.0). Moreover, we placed a weakly informative prior on the system initial conditions and the global coupling parameter K, as a normal distribution centered at the ground-truth with standard deviation of 1.0. The prior on the hyperparameters was considered as a generic weakly informative prior N(0, 1.0).

After fitting a Bayesian model, it is often necessary to measure the predictive accuracy of the inferred model. The information criteria and leave-one-out cross-validation(LOO) are two rigorous approaches to assess the model’s ability in prediction of new data. Taking the existing simulation draws from log-likelihood evaluated at the posterior of the parameter values, widely applicable information criterion (WAIC) and Pareto-smoothed importance sampling (PSIS) LOO allow for efficiently estimating predictive accuracy of a fitted Bayesian model within a negligible computational time relative to the cost of model fitting.

### Inference Diagnostics

After running a MCMC sampling algorithm, it is necessary to carry out some statistical analysis in order to evaluate the convergence of MCMC samples. One simple way to assess the performance of MCMC algorithms based on posterior samples is to visualize how well the chain is mixing (i.e., MCMC sampler explores all the modes in the parameter space efficiently). This can be monitored in different ways including traceplot (evolution of parameter estimates from MCMC draws over the iterations), pair plots (to identify collinearity between variables), and autocorrelation plot (to measure the degree of correlation between draws of MCMC samples). A more quantitative way to assess the MCMC convergence to the stationary distribution is to estimate the potential scale reduction factor R̂, and effective sample size N_{eff} based on the samples of posterior model probabilities. The R̂ diagnostic provides estimate of how much variance could be reduced by running chains longer. Each MCMC estimation has R̂ statistic associated with it, which is essentially the ratio of between-chain variance to within-chain variance. If R̂ is approximately less than 1.1, the MCMC convergence has been achieved (approaching to 1.0 in the case of infinite samples); otherwise, the chains need to be run longer. Moreover, the N_{eff} statistic gives the number of independent samples represented in the chain. The larger the effective sample size, higher the precision of MCMC estimates. Note that these are necessary but not sufficient conditions for convergence of MCMC samples.

In addition to the general MCMC diagnostics mentioned above, the NUTS-specific diagnostics can be used to monitor the convergence of samples; the number of divergent leapfrog transitions (due to highly varying posterior curvature), the step size used by NUTS in its Hamiltonian simulation (if the step size is too small, the sampler becomes inefficient, whereas if the step size is too large, the Hamiltonian simulation diverges), and the depth of tree used by NUTS, which is related to the number of leapfrog steps taken during the Hamiltonian simulation.

### Evaluate Posterior Fit

Using synthetic data for fitting allows us to validate the inference as the ground-truth of the parameters being inferred are known. Therefore, standard error metrics can be used to measure the similarity between the inferred parameters and those used for data generation. The metrics used to validate our inference are confusion matrix, posterior shrinkage, and posterior z-score.

Confusion matrix is a metric to evaluate the accuracy of a classification. The element q_{i,j} is equal to the number of observations known to be in class i but predicted to be in class j, with i, j ∈ {1, 2, ...Q}, where Q is the total number of classes. In the BVEP model, we defined three groups namely HZ, PZ, and EZ to classify brain regions, thus Q = 3.

Moreover, in order to quantify the accuracy of the inference, the posterior z-scores (denoted by z) were plotted against the posterior shrinkage (denoted by s), which are defined as:

$$z = \left|\frac{\overline{θ} - θ *}{{σ}_{p o s t}}\right| ,$$

$$s = 1 - \frac{{σ}_{p o s t}^{2}}{{σ}_{p r i o r}^{2}} ,$$

where θ̅ and θ* are the estimated-mean and the ground-truth, respectively, whereas

$${σ}_{p r i o r}^{2} \text{, and} {σ}_{p o s t}^{2}$$

indicate the variance (uncertainty) of the prior and the posterior, respectively. The posterior z-score quantifies how much the posterior distribution encompasses the ground-truth, while the posterior shrinkage quantifies how much the posterior distribution contracts from the initial prior distribution.

### Synthetic Data Sets and Model Inversion

In order to validate the inference using BVEP, advantage is taken of simulation capabilities of The Virtual Brain (TVB) for generating synthetic data sets. TVB is an open-source neuroinformatics tool written in Python to simulate large-scale brain network models based on individual subject data. This platform has been extensively used to simulate common neuroimaging signals including functional MRI (fMRI), EEG, SEEG and MEG with a wide range of clinical applications from Alzheimer disease, chronic stroke to human focal epilepsy (Jirsa et al., 2017).

In this study, TVB is used to reconstruct the personalized brain network model. In order to validate the inference on spatial epileptogenicity, epileptic seizures were simulated for two patients: one simulation with the seizure spread to all brain nodes specified as PZ (patient 1), and another with the seizure spread to some of the brain nodes specified as PZ (patient 2). These data sets were generated using two different structural connectivity matrices and distinct spatial map of epileptogenicity.

The seizure activity of patient 1 was simulated by setting two regions as EZ, and three regions as PZ, where EZ_{idx} ∈ {7,35}, and PZ_{idx} ∈ {6,12,28}, with the excitability values η_{ez} = –1.6, and η_{pz} = –2.4, respectively. All the other brain nodes were fixed as not epileptogenic i.e., HZ with η_{hz} = –3.6.

To simulate the seizure activity of patient 2, two brain regions were selected as EZ, and five regions as PZ, at the nodes EZ_{idx} ∈ {7, 24}, and PZ_{idx} ∈ {10,23, 27,28,35}, respectively. For the regions selected as EZ, the excitability value was set to η_{ez} = –1.5. The excitability of PZ was set as η_{pz} = –2,4, and all the other regions were defined as HZ with η_{hz} = –3.4.

In both synthetic data sets, to simulate the VEP model as a system of stochastic differential equations, an Euler-Maruyama integration scheme was used with an integration step of 0.04. The additive white Gaussian noise was introduced in the state variable x(t) = (x_{1,i}(t), y_{1,i}(t), zᵢ(t), x_{2,i}(t), y_{2,i}(t) with zero mean and variance (0.01, 0.01, 0.0, 0.0015, 0.0015). The initial conditions were selected in the interval (-2.0, 5.0) for each state variable.

Finally, to invert the BVEP for the simulated data sets, two popular open-source PPL tools were used for flexible probabilistic inference: Stan, and PyMC3. Stan language can be run in different interfaces, whereas PyMC3 provides several MCMC algorithms for model specification directly in native Python code. By specifying the model density functions in these tools, the gradients of functions are computed through automatic differentiation, a powerful technique for algorithmic computation of derivatives, to efficiently approximate the log-posterior density by NUTS and ADVI. The computation of independent MCMC chains can also be performed in parallel on separate processors. In this example, the Stan command line interface was used, whereas all the codes for simulations and posterior-based analysis were implemented in Python. The model simulation and parameter estimation were performed on a Linux machine with 3.0 GHz Intel Xeon processor and 32 GB of memory.

### Example 2: Results

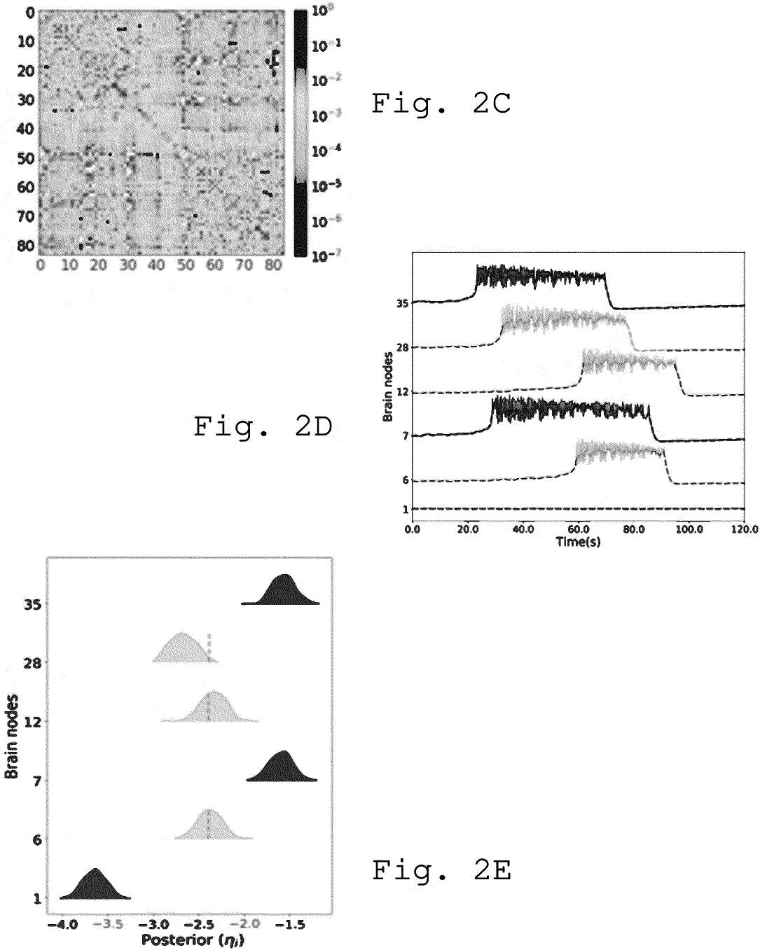

The result of workflow in the BVEP model to estimate the spatial map of epileptogenicity across different brain regions for patient 1 is illustrated in FIGS. 2A - 2E. Parcellation of the reconstructed brain and the patient’s brain network are shown in FIGS. 2A and B, respectively. Following Desikan-Killiany parcellation used in the reconstruction pipeline, the patient’s brain is divided into 68 cortical regions and 16 subcortical structures. FIG. 2C illustrates the structural connectivity matrix derived from diffusion tractography of the patient. Following the virtualization of the patient’s brain, TVB was used to simulate the reconstructed VEP brain network model. The simulated time series of fast activity variable in full VEP brain model are illustrated in FIG. 2D. The different brain node types namely HZ, PZ, and EZ are encoded in green, yellow and red, respectively. When the Epileptors are in isolation (i.e., K = 0; no network coupling), the seizures are triggered only in the regions defined as EZ, whereas no seizure propagation can be observed in other regions (see FIG. 2A and D). However, by coupling the Epileptors through the structural connectivity matrix of the patient (see FIG. 2C), the spatial recruitment pattern can be observed in the candidate brain regions defined as PZ (see FIG. 2D). In contrast to patient 2 (see FIG. 2F) where only one of the PZ is recruited, here, due to the strong coupling connections to regions specified as PZ as well as the high excitability value of these nodes, the seizure propagates to all other candidate brain regions specified as PZ (nodes number 6, 12, and 28). Average of fast activity variable inferred by inverting reduced VEP model is illustrated by the dashed line in FIG. 2D. It can be seen that there is a remarkable similarity between the simulated and the predicted seizures regarding the seizure initiation, propagation and termination. Note that the simulation illustrates the activity of fast variable in full VEP brain model (i.e., x_{1,i}(t) in Eq. (2)), whereas the inferred envelope of time series demonstrates the trajectories from inversion of reduced VEP model (cf. Eq. (3)). The estimated densities of the excitability parameters ηᵢ for different brain node types are shown in FIG. 2E. From this figure, it is observed that the true value of excitability parameter (dashed vertical line) is under the support of the estimated posterior density across different brain regions.

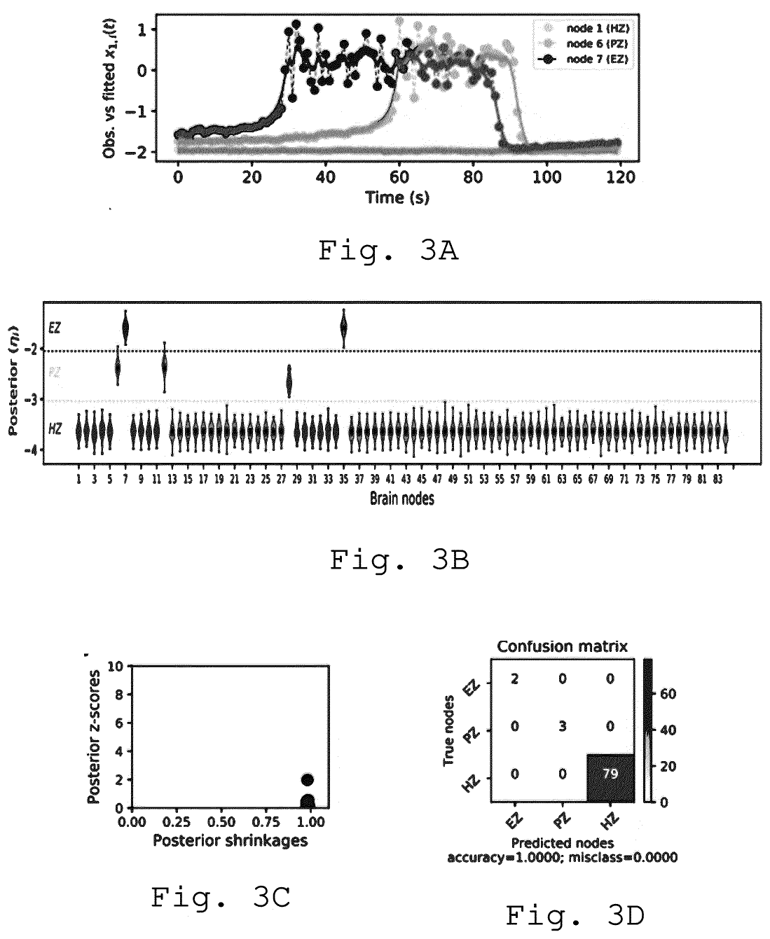

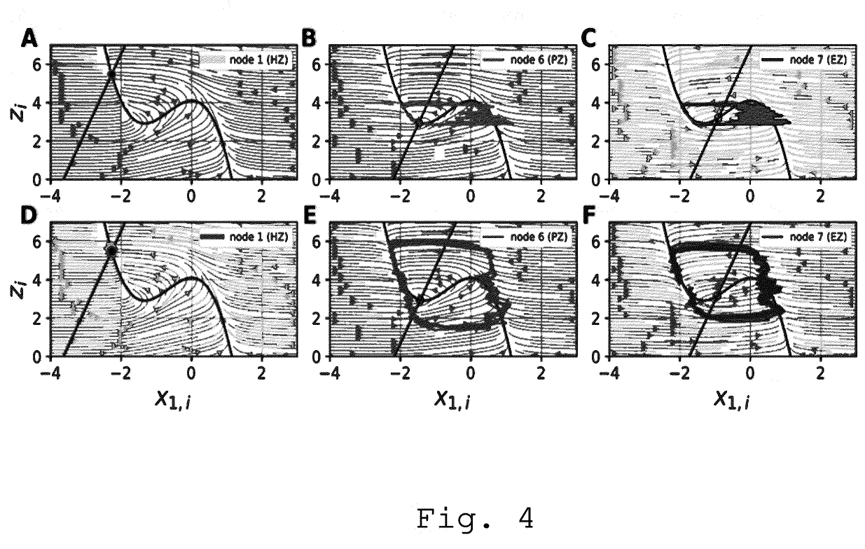

The accuracy of estimated spatial map of epileptogenicity across different brain regions for patient 1 by BVEP implementation in Stan is presented in FIGS. 3A to 3D. Similar result were obtained from BVEP implementation in PyMC3. FIG. 3A compares observed and inferred source activity for three brain node types specified as HZ, PZ, and EZ (nodes number 1, 6, 7, respectively). Simulated data consists of 120 s of activity of fast variable in full VEP brain model (i.e., x_{1,i}(t) in Eq. (2)) sampled at 1000 Hz, which is down-sampled by a factor of 10 to reduce the computational cost of the Bayesian inversion. The observed data is shown by dash-dotted line, whereas the shaded area illustrates the range between the 5th and 95th percentiles of the posterior predictive distribution. The activity of selected brain nodes in HZ, PZ, and EZ is shown in green, yellow and red, respectively. It is observed that the predicted time series based on the samples from the posterior predictive distribution are in very good agreement with the simulations. FIG. 3B shows the violin plot of the estimated density of the excitability parameter for all 84 brain regions included in the analysis. The filled black circles display true parameter values that were used to generate the simulated data. It can be seen that the ground-truth of excitability parameter for all brain areas is under the support of the estimated posterior distribution. As displayed in FIG. 3C, the distribution of posterior z-scores and posterior shrinkages for all the inferred excitabilities substantiates reliability of the model inversion. Note that the concentration towards large shrinkages indicates that all the posteriors in the inversion are well-identified, while the concentration towards small z-scores indicates that the true values are accurately encompassed in the posteriors. Therefore, the distribution on the bottom right of the plot implies an ideal Bayesian inversion. To further confirm the accuracy of the estimates in spatial excitabilities, the confusion matrix computed based on the inferred ηᵢ for i € {1, 2, ..., 84} is illustrated in FIG. 3D. The diagonal values in confusion matrix indicate that the pre-defined class for all the brain nodes labeled as HZ, PZ, and EZ are accurately predicted (accuracy=1.0, misclassification=0.0).

In order to investigate whether the BVEP is a platform-independent framework, we also used PyMC3 to estimate the spatial map of epileptogenicity across different brain regions. For both patients analyzed, the same accuracy was obtained by inversion of Eq. 4 in Stan and PyMC3. These results indicate that the BVEP inversion in Stan and PyMC3 leads to similar estimation of spatial map of epileptogenicity across brain regions in both analyzed patients.

Furthermore, the NUTS-specific diagnostics were monitored to check whether the Markov chain has converged. The diagnostics plot shows that there are no divergent transitions in HMC indicating that the posterior density was explored efficiently. Also, none of the NUTS iterations reached maximum tree-depth (its value to run NUTS was specified 10.0 here) indicating that the optimal number of leapfrog steps needed for the Hamiltonian simulation was sufficiently lower than the maximum. Together, these diagnostics validate that the samples by NUTS has converged to the target distribution.

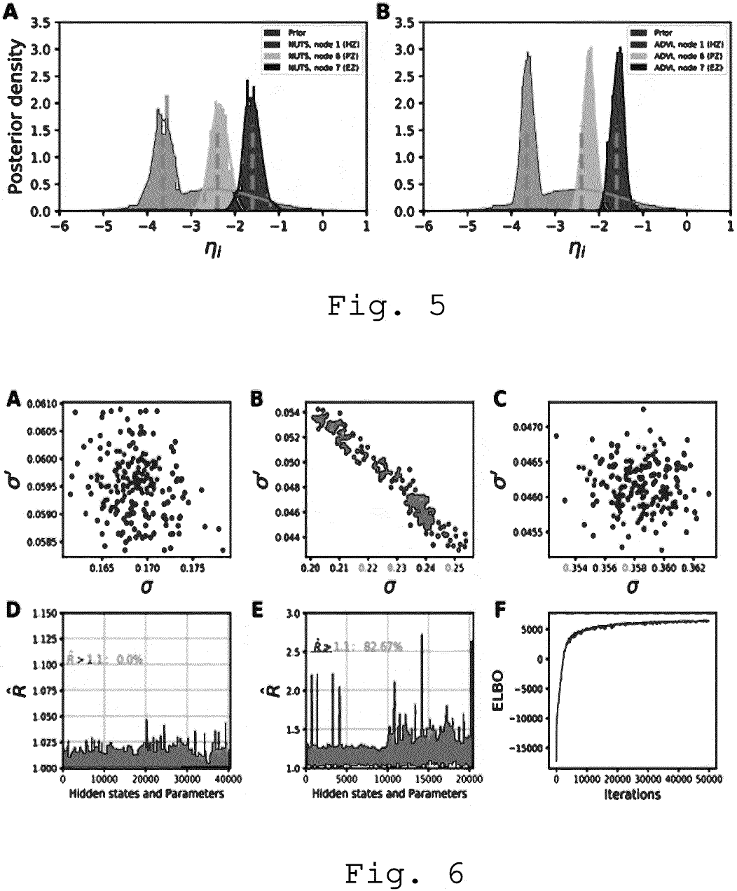

To illustrate the mechanisms underlying seizure initiation and propagation within the BVEP model, the phase-plane topology of the simulation (top row) versus the prediction (bottom row) characterizing the dynamics of the different brain node types in the BVEP model is presented in FIG. 4. In the plotted phase-planes, the x- and z-nullclines are colored in dark grey, where the intersection of the nullclines identifies the fixed point of the system. From left to right, the columns correspond to the brain nodes specified as HZ, PZ, and EZ, respectively. Full circle and empty circle indicate the stable and unstable fixed points, respectively. From FIGS. 4A and D, it can be observed that a trajectory of an HZ (node number 1) is attracted to the stable fixed point of the system (on the left branch of cubic x-nullcline) meaning not triggering epileptic seizure. For a PZ (node number 6), due to the coupling strength and the value of excitability which is close to the critical value of epileptogenicity, the z-nullcline moves down, causing a bifurcation thereby allowing the seizure to propagate here (see FIGS. 4B and E). For the EZ (node number 7), the system exhibits an unstable fixed point due to the high value of excitability. In this regime, Epileptor possesses a limit cycle and the seizure triggers autonomously (see FIGS. 4C and F). Notice that the topology of simulated and predicted phase-plane trajectories show very good agreement, except the amplitude of state variable zᵢ, from which the estimation indicates the result of a larger parameter recovery. Note that only the activity of fast variable x_{1,i} is the target of fitting as the observed data.

To compare the BVEP inversion by NUTS and ADVI schemes, FIG. 5 displays the histogram of MCMC samples and the kernel density estimates of the posteriors generated from NUTS (left panel) versus those obtained by ADVI (right panel). From this figure, it is observed that NUTS and ADVI perform similarly in their estimates of the posterior, except that the mean-field ADVI slightly underestimates the variances compared to the estimations by NUTS algorithm. However, taking both approaches, the true values of excitabilities (dashed vertical lines) are under the support of the posterior densities indicating that the parameter recovery was successful. The samples corresponding to the brain nodes specified as HZ, PZ, and EZ are illustrated in grey, light grey and dark grey, respectively. Note that the prior for all 84 brain regions included in the analysis was assumed as a normal distribution centered at –2.5 with standard deviation of 1.0 (i.e., N(-2.5, 1.0) as shown in blue. To invert the BVEP model by NUTS algorithm, 200 number of sampling iterations and 200 warmup were used with the expected acceptance probability of 0.95, whereas to run ADVI, the maximum number of iterations and the convergence tolerance were set to 50000 and 0.001, respectively. In terms of computational time, for these algorithm configurations, sampling by NUTS took 23993.5 seconds whereas the running time of ADVI was 5392.62 seconds.

Once the model parameters have been estimated, it is necessary to assess the convergence of MCMC samples. To verify the reliability of the inferred estimates, we monitored the potential scale reduction factor R̂ as it is the most reliable quantitative metric for MCMC convergence. In addition, the posterior samples from the joint posterior probability distribution were plotted to show the efficiency of the transformed non-centered parameterization in comparison to the centered form of parameterization. FIG. 6 top row represents the posterior samples from the joint posterior probability distribution between the hyper-parameters σ and σ′, which are the standard deviation of the process (dynamical) noise and the measured noise, respectively (cf., Eq. (4)). In this figure, the left and middle columns show the result of sampling by NUTS with non-centered and centered form of parameterization, respectively. For the sake of comparison with NUTS, the last column illustrates the result from mean-field variant of ADVI. The dots in each scatterplot represent 200 samples drawn from the joint posterior probability distribution. In FIGS. 6A and B, it can be clearly seen that there is no correlation between the posterior samples drawn from non-centered parameterization, whereas the samples from the centered form show a high collinearity between hyper-parameters. Such a high collinearity leads to inefficient exploration of posterior which can be quantifiably observed in decreased numbers of effective samples and increased R̂ values. The values of R̂ for all of hidden states and parameters estimated by non-centered form are below 1.05 (see FIG. 6D), whereas more than 82 percent estimations by centered form has R̂ value above 1.1 (see FIG. 6E). This indicates that the Markov chains converged for non-centered but not for centered form of parameterization. The ratio of effective number of samples to the number of iteration (N_{eff}=Nᵢₜₑᵣ) returned by centered form of NUTS is less than 0.001 for all the estimated parameters. This indicates the poor sampling from centered form of parameterization as it generates a very small number of independent samples per Markov chain.

Moreover, scatterplot of samples drawn from joint posterior probability distribution between the hyper–parameters σ and σ′ estimated by the mean-field ADVI is illustrated in FIG. 6C. Since by definition, the mean-field variant of ADVI ignores the cross correlation between parameters, the samples drawn using mean-field ADVI show no correlation between hyper-parameters. Lastly, in order to check the convergence of ADVI, evidence lower bound (ELBO), the variational objective function, is plotted versus the number of iterations (see FIG. 6F). While the algorithm appears to have converged in 10000 iterations, the algorithm runs for another few thousand iterations to guarantee the convergence until the change in ELBO drops below the tolerance of 0.001.

Finally, this invention presents a probabilistic framework namely the Bayesian Virtual Epileptic Patient (BVEP) to infer the spatial map of epileptogenicity for developing a personalized large-scale brain model of epilepsy spread (cf. FIG. 1). The workflow to build the BVEP brain model consists of two main steps: in the first step, the VEP is constructed i.e., the personalized large-scale brain network model of epilepsy spread. In the VEP model, the dynamics of brain nodes are governed by the neural population model of epilepsy namely Epileptor, which is a generic model to realistically reproduce the onset, progression and offset of seizure patterns across species and brain regions (Jirsa et al., 2014). The Epileptors are coupled through the patient’s connectome to combine the mean-field model of abnormal neuronal activity with the subject-specific brain’s anatomical information derived from non-invasive diffusion neuroimaging techniques (MRI, DTI). Together with patient’s data, the VEP model was then furnished with the spatial map of epileptogenicity across different brain regions. In the second step, the VEP was embedded as the generative model in PPL tools (Stan/PyMC3) to infer and validate the spatial map of epileptogenicity across different brain regions. Using the PPLs along with high-performance computing to run several MCMC chains in parallel enables systematic and efficient parameter inference to fit and validate the BVEP model against the patient’s data.

To demonstrate the potential functionality of the BVEP in prediction of seizure initiation and propagation, simple and complex seizure spread were simulated using different spatial maps of epileptogenicity (cf. FIG. S2). These synthetic data were used for fitting, since given the ground-truth of model parameters, the standard error metrics can be used such as confusion matrix, posterior shrinkages and posterior z-scores to validate the accuracy of the estimations, thus to evaluate the performance of the proposed approach. The results demonstrated that in both synthetic data sets, by inverting the large-scale brain network model with the help of PPLs (Stan/PyMC3), it can achieve a remarkable similarity between the simulated and the predicted seizure activity regarding the initiation, propagation and termination. Although, the simulation was generated by the full VEP model comprising five state variables at each brain node, the 2D reduced variant of the model was still able to successfully predict the key data features such as onset, propagation and offset of seizure patterns, while considerably alleviating the computational time of the Bayesian inference. This 2D reduction is limited to modeling the average of fast discharges during the ictal seizure states, which as shown (see FIG. 3, and S5) is a sufficient feature for correctly estimating the spatial map of epileptogenicity. The results indicated that the BVEP model is able to accurately estimate the spatial map of epileptogenicity across different brain regions (cf. FIGS. 3A-3D). The true value of excitability for all brain nodes included in analysis was under the support of estimated posterior densities with 100% classification accuracy based on the confusion matrix. In addition, the concentration of distribution toward small z-scores along with concentration towards large posterior shrinkages (i.e., the bottom right corner in FIG. 3C) confirmed the reliability of model inversion. Note that relying on the accuracy obtained by confusion matrix may be inconclusive, since the accuracy of estimation return by this metric depends only on the mean value of estimated posterior densities. For instance, consider inferences where the mean of posteriors are nearly identical to the ground-truth, however there is a large uncertainty over the estimations. In such cases, confusion matrix may result in a high accuracy performance, whereas plotting the posterior z-scores versus the posterior shrinkages is particularly useful for identifying the malfunctioning in the inference such as overfitting, or a poorly-chosen prior that biases the estimations.

Understanding brain dynamics in epilepsy is critical for developing therapeutic approaches towards brain interventions to improve the surgical outcome. Using theory of nonlinear dynamic systems, the complete taxonomy of epileptic seizures with a thorough description of bifurcations that give rise to onset, offset and seizure evolution characteristics has been extensively investigated elsewhere (Jirsa et al., 2014). In parameter space description of Epileptor, the seizure onset and offset are described by saddle-node and homoclinic bifurcations. The emergent dynamic effects in the BVEP model crucially depend on the interplay between network node model (Epileptor), patient specific structural connectivity (from dMRI), and spatial maps of epileptogenicity (EZ, PZ, HZ). According to the dynamical properties of Epileptor model, the brain regions were classified into three main types: EZ (exhibiting unstable fixed point corresponding to the brain area responsible for the seizure initiation), PZ (close to saddle-node bifurcation corresponding to the candidate brain area responsible for the seizure propagation), and HZ (exhibiting stable fixed point corresponding to healthy brain area). This approach allows us to define the spatial map of epileptogenicity based on the excitability parameter value, which is the target of fitting.

It is important to note that an excitability value close to the critical value of epileptogenicity does not guarantee that the seizure originating from pathological brain areas (i.e., responsible for the seizure onset specified as EZ) propagates to such brain regions defined as PZ. By a detailed patient evaluation, it has been reported that the individual structural connectivity is essential for predicting seizure spatial propagation. However, it has been recently shown that purely structural information is not sufficient to predict the propagation and eventual stopping of the seizures. Rather, the abnormal activity in the recruited regions is a complex network effect which depends on the interplay between multiple factors including the brain region’s epileptogenicity (node dynamics), the individual structural connectivity (network structure) (Jirsa et al., 2017), and brain state dependence (network dynamics). Furthermore, there are nonlinearities and multiple propagation patterns that can be observed for the same excitability parameter sets due to the coupled nonlinear system dynamics (cf., FIG. S2). In this work, the seizure recruitment is characterized by complex spatiotemporal dynamics of large-scale brain network i.e., seizure originates from a local network and recruits candidate brain regions strongly coupled to the pathological areas by perturbing their stable dynamics (if K=0, then there is no seizure recruitment). Among the candidate brain regions for seizure propagation, due to stronger connection to the pathological areas defined as EZ, the node PZ_{idx} = {28} can be recruited by a weak global coupling. Rather, a stronger coupling is required for seizure recruitment to all other candidate brain areas. This is in agreement with experimental observations that seizures tend to have a common spatial origin in the same patient. According to this knowledge, it was placed a weakly informative prior on the global coupling parameter centered at the ground-truth. Overestimation of global coupling parameter leads to miss-classification of PZ as HZ (see FIGS. 2E and 3B), whereas an underestimation of coupling may yield to miss-classification of PZ as EZ. However, the stability analysis of the network dynamics indicates that the seizure propagation can be controlled by an optimal intervention on the structural connectivity matrix implying that the patient-specific network connectivity is predictive for seizure propagation pattern. Therefore, the seizure propagation may not be easily controlled by a simple dissection of the individual nodes, as in the surgical treatment of epilepsy, it has been reported that the resection does not always lead to post-surgery seizure freedom in the brain.

In this study, the analysis of phase-plane trajectories of the observed system versus the prediction was carried out across different brain regions in order to gain a better understanding of the mechanisms underlying seizure initiation and propagation within the proposed approach (cf. FIG. 4). For different brain node types (e.g., EZ, PZ, and HZ), the dynamics of seizure initiation and recruitment in the phase-plane was captured well by the prediction. From inference perspective, a good correspondence was observed to the phase portraits of the observed system including equilibria (the intersection of the nullclines), the stability or instability of the equilibria, and the flow of trajectories. These results validate our Bayesian inversion procedure in order to understand the spatiotemporal evolution of seizure activity, paving the way for further studies on possible seizure prevention approaches.

Both NUTS and ADVI schemes were used to infer the spatial map of epileptogenicity in a personalized whole-brain model of epilepsy spread. The results from both inference schemes led to similar estimation of spatial map of epileptogenicity across brain regions, except that the ADVI slightly underestimate the variances compared to the estimations by NUTS algorithm (cf. FIG. 5). The similarity between the inversions using the two schemes indicates that the variational approximation offer an appropriate alternative to NUTS sampling in BVEP model inversion. Our results demonstrated that there is a clear reduction in computational cost in performing the inference by ADVI compared to NUTS (4-5 times faster for the used algorithm configurations), which may be important when applying BVEP approach to large data sets of patient cohort. While it is generally known that ADVI is more computationally appealing than NUTS, however, it can be challenging to discover algorithmic problems with this approximation. The convergence of ADVI can be assessed by monitoring the running average of ELBO changes, whereas NUTS is furnished with several general and specific diagnostics to assess whether the Markov chain has converged. In addition, ADVI may get stuck in local minima during gradient descent optimization and its mean-field variant is unable to cover all the modes of the multi-modal posterior densities.

Lastly, the efficiency of transformed non-centered parameterization was investigated. In agreement with previous studies showing that NUTS is sensitive to the parameterization, our results indicated that the non-centered form of parameterization to invert the nonlinear state-space equations yields an efficient parameter-space exploration, whereas the centered form of sampling demonstrates an inefficient exploration due to the high collinearity between model parameters (see FIGS. 6A and D versus FIGS. 6B and E). In addition, based on the convergence diagnostics such as R̂, we demonstrated that the samples generated by NUTS converged faster in the non-centered parameterization compared to the centered form of parameterization.

A novel approach to build personalized in-silico brain network models based on Bayesian inference within PPL tools such as Stan and PyMC3. Although several PPL libraries have been developed for Bayesian inference, only a few of them are built around efficient sampling algorithms such as NUTS that avoids the random walk behavior and sensitivity to correlated parameters. Both Stan and PyMC3 provide NUTS and ADVI with automatic differentiation to efficiently compute gradients without requiring user intervention. Stan is a generic and flexible software package that has interfaces for common data science languages, also providing extensive diagnostics for MCMC convergence. PyMC3 provides several MCMC algorithms by model specification directly in native Python code. Our implementation in both Stan and PyMC3 result in similar estimation of spatial map of epileptogenicity across brain regions indicating that BVEP is a platform-independent approach. However, a larger number of warm-up iterations were required in PyMC3 to arrive at the same posterior convergence achieved by our implementation in Stan. This is due to the differences in implementation of NUTS in Stan and PyMC3. Comparison of the implementations in Stan, PyMC3 and other alternative PPL packages is beyond the scope of this note.

This invention is the first personalized large-scale brain network modeling approach for inferring the spatial map of epileptogenicity (properties of nodes) based on patient-specific whole-brain anatomical information (i.e., network structure derived from dMRI). Dynamic Causal Modelling (DCM) is a well-established framework for analyzing neuroimaging modalities (such as fMRI, MEG, and EEG) by neural mass models where inferences can be made about the coupling among brain regions (effective connectivity) to infer how the changes in neuronal activity of brain regions are caused by activity in the other regions through the modulation in the latent coupling. Using DCM, focal seizure activity in electrocorticography (ECoG) data was recently studied to estimate the key synaptic parameters or coupling connections using observed signals in a human subject. In another study, Bayesian belief updating scheme for DCM has been used to estimate the synaptic drivers of cortical dynamics during a seizure from EEG/ECoG recordings with a little computational expense. Although DCM can be used to model and track the changes in excitatory{inhibitory balance at seizure onset/offset, these studies are based on single neural mass model (i.e., small number of cortical sources are modelled), and the non-linear ordinary differential equation representing the neural mass model is approximated by its linearization, with which only the seizure onset or offset can be modelled but not both. In this note, the Bayesian Virtual Epileptic Patient (BVEP) model can characterize whole-brain spatiotemporal nonlinear dynamics of seizure propagation. This approach allows describing the onset and offset of ictal states as well as the alternation between normal and ictal periods. The BVEP approach relies on the patient-specific structural data rather formulating the inverse problem purely in terms of unknown model parameters used in DCM. It is also worth mentioning that the dynamics of system was inferred with coupled fast and slow time-scales (cf., Eq. (3)), therefore, the variations in slow variable depend on the hidden states of fast activity while it is assumed that only the activity of fast variable is observed. In this study, the time-scale separation in Epileptor model enabled to capture reliably full evolutions of complex dynamics, ranging from pre-ictal to onset, ictal evolution and offset rather using time-varying parameters. Future extensions to the current work could examine explicitly the non-stationary dynamics of networks in order to investigate conditions for mechanism of seizure initiation whether the seizure onset is more likely to occur through a deterministic parameter changes as in a bifurcation or it is a jump phenomenon due to the noise-driven transition between bistable attractors. The Bayesian inversion in the current work is based on the auto-tuning algorithms such as NUTS and ADVI accomplished with fast automatic differentiation for the calculation of gradients. This allows to efficiently sample from complex and high-dimensional posterior distributions with correlated parameters compared to the traditional sampling algorithms. The inferences in the presented framework is also enriched with several MCMC convergence diagnostics to assess the reliability of the estimations.

Various noninvasive and invasive methods have been used to improve pre-surgical evaluation in identification of the EZ, and consequently to increases surgery success rates. Employing the BVEP model in clinical therapies and brain interventions will require quantification of the model outcomes in fitting empirical secondary functional signals of patients such as EEG, MEG, SEEG, and fMRI signals. In this framework, it is straightforward to incorporate further knowledge such as MRI lesions and clinical hypothesis on EZ from pre-surgical evaluation. Since the BVEP model can be considered as a generic approach towards large-scale brain modeling, it offers promising avenue for inference from clinically used non-invasive imaging signals (EEG, MEG, fMRI), and invasive measurements such as SEEG signals. The results indicate that the proposed approach according to this invention is able to successfully fit against the patient’s empirical SEEG data (not shown). Note that in the case of empirical SEEG recording, the source localization is an ill-posed problem due to the sparsity of lead-field matrix, which can affect the accuracy of the estimates. In principle, it is possible that the surgical strategies can be systematically tested using the BVEP model, however, the real clinical application remains to be investigated and validated in future work.

In conclusion, the invention establishes a link between the probabilistic modeling and personalized brain network modeling in order to systematically predict the location of seizure initiation in a virtual epileptic patient. It is demonstrated step by step, how the proposed framework allows one to infer the spatial map of epileptogenicity based on large-scale brain network models that are derived from noninvasive structural data of individual patients. The invention rests on advanced efficient sampling algorithms that provide accurate and reliable estimates validated by the posterior behavior analysis and convergence diagnostics. In summary, with the help of PPLs, the use of personalized brain network models offer a proper guidance for development of comprehensive clinical hypothesis testing and novel surgical intervention.

1. A method for inferring epileptogenicity of a brain region that is not observed as recruited or is not observed as not recruited, in a seizure activity of an epileptic patient brain, comprising:
providing a computerized model modelling various regions of a primate brain and connectivity between the regions;
providing the computerized model with a model able to reproduce an epileptic seizure dynamic in the primate brain, the model being a function of a parameter that is the epileptogenicity of a region of the brain;
providing structural data of the epileptic patient brain and personalizing the computerized model using the structural data in order to obtain a virtual epileptic patient (VEP) brain model;
translating a state-space representation of the virtual epileptic patient (VEP) brain model into a probabilistic programming language (PPL) using probabilistic state transitions in order to obtain a probabilistic virtual epileptic patient brain model (BVEP); and
acquiring electro- or magneto- encephalographic data of the patient brain and fitting the probabilistic virtual epileptic patient brain model against the data in order to infer the epileptogenicity of the brain region that is not observed as not recruited or is not observed as not recruited, in the seizure activity of the patient brain.

2. The method according to claim 1, wherein the probabilistic programming language is a Bayesian programming language, the probabilistic virtual epileptic patient brain model is Bayesian virtual epileptic patient (BVEP) brain model, and the epileptogenicity of the brain region that is not observed as recruited or is not observed as not recruited is inferred using Bayesian inference.

3. The method according to claim 1, wherein the structural data of the epileptic patient brain comprise non-invasive T1-weighted imaging data and/or diffusion MRI images data.

4. The method according to claim 1, wherein the model able to reproduce the epileptic seizure dynamic in the primate brain is a model which reproduces the dynamics of an onset, a progression and an offset seizure events, that comprises state variables coupling two oscillatory dynamical systems on three different timescales, a fastest timescale wherein state variables account for fast discharges during an ictal seizure state, an intermediate timescale wherein state variables represent slow spike-and-wave oscillation, and a slowest timescale wherein a state variable is responsible for the transition between interictal and ictal states, and wherein a degree of epileptogenicity of a region of the brain is represented through a value of an excitability parameter.

5. The method according to claim 1, wherein, for obtaining the probabilistic virtual epileptic patient brain model, a spatial map of epileptogenicity of the patient brain is provided, the spatial map of epileptogenicity classifying brain regions of the patient brain into epileptogenic zones (EZ) which can trigger epileptic seizures autonomously, propagation zones (PZ) which do not trigger seizures autonomously but can be recruited during a seizure evolution, and healthy zones (HZ) that do not trigger seizures autonomously.

6. The method according to claim 1, wherein the probabilistic virtual epileptic patient brain model is generated according to a generative model based on the state-space representation of the virtual epileptic patient.

7. The method according to claim 6, wherein the state-space representation of the virtual epileptic patient is of the form:
$$\left\{\begin{matrix}\dot{x} \left(t\right) = f \left(x \left(t\right) , u \left(t\right) , θ\right) + w \left(t\right) , x \left(0\right) = {x}_{{t}_{0}} \\ y \left(t\right) = h \left(x \left(t\right)\right) + v \left(t\right)\end{matrix}\right)$$
where
x(t) ∈ ℝⁿ is a n-dimensional vector of system’s states evolving overtime,
^{x}t₀ is an initial state vector at time t = 0,
θ ∈ ℝ^{p} contains all the unknown parameters of the virtual epileptic patient model,
u(t) stands for the external input,
y(t) ∈ ℝ^{m} denotes the measured data subject to the measurement error v(t),
f is a vector function that describes dynamical properties of the system, and
h represents a measurement function.

8. The method according to claim 1, wherein, for obtaining of the probabilistic virtual epileptic patient (BVEP) model, the state-space representation of the virtual epileptic patient (VEP) model is incorporated in the probabilistic virtual epileptic patient (BVEP) model as state transition probabilities.

9. The method according to claim 7,
wherein, for obtaining of the probabi listic virtual epileptic patient (BVEP) model, the state-space representation of the virtual epileptic patient (VEP) model is incorporated in the probabilistic virtual epileptic patient (BVEP) model as state transition probabilities, and
wherein the state transition probabilities are:
$$J \left(x \left(t\right)\right) , x \left(t + 1\right) ∼ N \left(x \left(t\right) + d t f \left(x \left(t\right) , u \left(t\right) , θ\right) , {σ}^{2}\right)$$
where τ denotes a transition probability from a state x(t) to x(t + dt).

10. The method according to claim 6, wherein the generative model is defined in terms of likelihood and prior on model parameters, whose product yields a joint density:
$$P \left(y , ϑ\right) = P \left(y \vert ϑ\right) P \left(ϑ\right)$$
where prior distribution P(ϑ) includes our prior beliefs about the hidden variables and potential parameter values, while the conditional likelihood term P(y|ϑ) represents the probability of obtaining an observation, with a given set of parameter values.

11. The method according to claim 1, comprising implementing at least one sampling algorithm in order to infer the epileptogenicity of the brain region that is not observed as recruited or not observed as not recruited, in the seizure activity of the patient brain.

12. The method according to claim 11, wherein the at least one sampling algorithm is a Markov chain MMonte Carlo or a variational inference algorithm.

13. The method according to claim 1, wherein the method is implemented on a computer.

14. The method according to claim 2, wherein the structural data of the epileptic patient brain comprise non-invasive T1-weighted imaging data and/or diffusion MRI images data.

15. The method according to claim 14, wherein the model able to reproduce the epileptic seizure dynamic in the primate brain is a model which reproduces the dynamics of an onset, a progression and an offset seizure events, that comprises state variables coupling two oscillatory dynamical systems on three different timescales, a fastest timescale wherein state variables account for fast discharges during an ictal seizure state, an intermediate timescale wherein state variables represent slow spike-and-wave oscillation, and a slowest timescale wherein a state variable is responsible for the transition between interictal and ictal states, and wherein a degree of epileptogenicity of a region of the brain is represented through a value of an excitability parameter.

16. The method according to claim 2, wherein the model able to reproduce the epileptic seizure dynamic in the primate brain is a model which reproduces the dynamics of an onset, a progression and an offset seizure events, that comprises state variables coupling two oscillatory dynamical systems on three different timescales, a fastest timescale wherein state variables account for fast discharges during an ictal seizure state, an intermediate timescale wherein state variables represent slow spike-and-wave oscillation, and a slowest timescale wherein a state variable is responsible for the transition between interictal and ictal states, and wherein a degree of epileptogenicity of a region of the brain is represented through a value of an excitability parameter.

17. The method according to claim 3, wherein the model able to reproduce the epileptic seizure dynamic in the primate brain is a model which reproduces the dynamics of an onset, a progression and an offset seizure events, that comprises state variables coupling two oscillatory dynamical systems on three different timescales, a fastest timescale wherein state variables account for fast discharges during an ictal seizure state, an intermediate timescale wherein state variables represent slow spike-and-wave oscillation, and a slowest timescale wherein a state variable is responsible for the transition between interictal and ictal states, and wherein a degree of epileptogenicity of a region of the brain is represented through a value of an excitability parameter.

18. The method according to claim 2, wherein, for obtaining the probabilistic virtual epileptic patient brain model, a spatial map of epileptogenicity of the patient brain is provided, the spatial map of epileptogenicity classifying brain regions of the patient brain into epileptogenic zones (EZ) which can trigger epileptic seizures autonomously, propagation zones (PZ) which do not trigger seizures autonomously but can be recruited during a seizure evolution, and healthy zones (HZ) that do not trigger seizures autonomously.

19. The method according to claim 3, wherein, for obtaining the probabilistic virtual epileptic patient brain model, a spatial map of epileptogenicity of the patient brain is provided, the spatial map of epileptogenicity classifying brain regions of the patient brain into epileptogenic zones (EZ) which can trigger epileptic seizures autonomously, propagation zones (PZ) which do not trigger seizures autonomously but can be recruited during a seizure evolution, and healthy zones (HZ) that do not trigger seizures autonomously.

20. The method according to claim 4, wherein, for obtaining the probabilistic virtual epileptic patient brain model, a spatial map of epileptogenicity of the patient brain is provided, the spatial map of epileptogenicity classifying brain regions of the patient brain into epileptogenic zones (EZ) which can trigger epileptic seizures autonomously, propagation zones (PZ) which do not trigger seizures autonomously but can be recruited during a seizure evolution, and healthy zones (HZ) that do not trigger seizures autonomously.
